(19) United States
(12) Patent Application Publication
(10) Pub. No.: US 2011/0245163 A1
(43) Pub. Date: Oct. 6, 2011

(54) Stabilised Insulin Compositions

(75) Inventors: Ib Jonassen, Valby (DK); Sven Havelund, Bagsvaerd (DK); Thomas Børglund Kjeldsen, Virum (DK); Ulla Ribel-Madsen, Virum (DK)

(73) Assignee: Novo Nordisk A/S, Bagsvaerd (DK)

(21) Appl. No.: 13/080,162

(22) Filed: Apr. 5, 2011

Related U.S. Application Data
(63) Continuation of application No. 12/476,712, filed on Jun. 2, 2009, now abandoned.
Application No. 12/476,712 is a continuation of application No. 11/328,606, filed on Jan. 10, 2006, now abandoned.
Application No. 11/328,606 is a continuation of application No. PCT/DK2004/000481, filed on Jul. 5, 2004.
(60) Provisional application No. 60/487,683, filed on Jul. 16, 2003.

(30) Foreign Application Priority Data
Jul. 11, 2003 (DK) PA 2003 01065

(51) Int. Cl. A61K 38/28; C07K 14/62; A61P 3/10

(52) U.S. Cl. 514/5.9; 530/303

(57) ABSTRACT

The present invention relates to human insulin analogues having a fast onset of action. These analogues may have amino acid in position B26 substituted with Phe, or be Des(B30) analogues of human insulin. The invention also relates to compositions comprising such insulin analogues, and to compositions comprising a mixture of an insulin analogue having a fast onset of action and insulin having a protracted action.

### CROSS-REFERENCE TO RELATED APPLICATIONS

This application is a continuation of U.S. patent application Ser. No. 12/476,712, field Jun. 2, 2009, which is a continuation of U.S. patent application Ser. No. 11/328,606, filed Jan. 10, 2006, which is a continuation of International Application No. PCT/DK2004/000481, filed Jul. 5, 2004, which claims priority from Danish Patent Application No. PA 2003 01065 filed Jul. 11, 2003 and to U.S. Patent Application No. 60/487,683 filed Jul. 16, 2003; the contents of all above-named applications are incorporated herein by reference.

### FIELD OF THE INVENTION

The present invention relates to insulin analogues having a fast onset of action. The invention also relates to compositions comprising such insulin analogues, and to compositions comprising a mixture of an insulin analogue having a fast onset of action and insulin having a protracted action.

### BACKGROUND OF THE INVENTION

Human insulin is a 51 amino acid peptide hormone consisting of an A-chain and a B-chain having 21 and 30 amino acid residues respectively, interconnected by two cysteine bridges. Insulin may aggregate into hexamers, in which form the hormone is protected from chemical and physical degradation during synthesis and storage. The action of such insulin hexamers is delayed because the hexamers must diffuse and dissociate into dimmers and monomers.

Many diabetic patients are treated with multiple daily insulin injections in a regimen comprising one or two daily injections of a protracted insulin to cover the basal requirement supplemented by bolus injections of a rapid acting insulin to cover the requirement related to meals.

Protracted insulin compositions are well known in the art. Thus, one main type of protracted insulin compositions comprises injectable aqueous suspensions of insulin crystals or amorphous insulin. In these compositions, the insulin compounds utilized typically are protamine insulin, zinc insulin or protamine zinc insulin. Certain drawbacks are associated with the use of insulin suspensions, e.g. the need to suspend the insulin particles by gentle shaking before a defined volume of the suspension is withdrawn from a vial or expelled from a cartridge

Another type of protracted insulin compositions are solutions having a pH value below physiological pH from which the insulin will precipitate because of the rise in the pH value when the solution is injected. A drawback with these solutions is that the particle size distribution of the precipitate formed in the tissue on injection, and thus the timing of the medication, depends on the blood flow at the injection site and other parameters in a somewhat unpredictable manner. A further drawback is that the solid particles of the insulin may act as a local irritant causing inflammation of the tissue at the site of injection.

A further type of protracted insulin compositions is those in which the ε-amino group of residue Lys^{B29} has been acylated with a long-chain fatty acid, see e.g. WO 95/07931 and WO 98/02460 (Novo Nordisk A/S). One such soluble insulin derivative is B29-N^{ε}-(N-lithocholyl-γ-glutamyl)-des(B30) human insulin. The protracted action has been explained by a reversible binding to albumin in subcutis, blood and peripheral tissue (see e.g. Markussen, Diabetologia 39, 281-288, 1996).

Rapid-acting insulin analogues are known, in which a mutation has been introduced with the aim of reducing the tendency to associate into higher molecular weight forms. Examples of such analogues are Asp^{B28} and Lys^{B28}Pro^{B29} human insulin.

Some patients use insulin compositions having both a fast onset of action and a more prolonged action. This is effected by using an insulin composition comprising two types of insulin, where one has a fast onset of action and the other a more prolonged action. The two types of insulin may be present in different ratios. Such compositions may be prepared by the patients themselves prior to injection, or more conveniently the composition may be pre-mixed and ready for injection. In such pre-mixed compositions the exchange of insulin monomers between the rapid-acting insulin analogue and the prolonged-action insulin hexamer may take place, which may result in a inferior release profile of insulin.

There is thus a need for insulin analogues suitable for pre-mixed compositions which retain the action profiles of both insulin components.

### SUMMARY OF THE INVENTION

The invention provides analogues of human insulin wherein the amino acid in position B26 is Phe. The remaining amino acid sequence may be identical to that of human insulin or may contain substitutions or deletions.

The invention also provides a pharmaceutical preparation comprising
i. A derivative of human insulin
ii. an analogue of human insulin having a fast onset of action, wherein
   Phe(B1) is deleted, or
   Tyr(B26) is replaced with Phe, or
   Phe(B1) is deleted and Tyr(B26) is replaced with Phe.

Also provided by the invention is a pharmaceutical preparation wherein the ratio of the derivative of human insulin i. to the analogue of human insulin having a fast onset of action ii. is between 1:99 and 99:1, such as between 10:90 and 90:10, e.g. between 30:70 and 70:30. The derivative of human insulin i. may be selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin, such as B29-N^{ε}-(N-lithocholyl-γ-glutamyl)-des(B30) human insulin, e.g. B29-N^{ε}-myristoyl-des(B30) human insulin.

The rapid acting component ii. may be Des(B1) human insulin or Des(B1) Des(B30) human insulin, such as Des(B1) human insulin.

Also provided by the invention is a pharmaceutical preparation which further comprises a phenolic preservative.

Also provided by the invention is a method of treating type 1 or type 2 diabetes comprising administering to a patient in need thereof a therapeutically effective amount of a pharmaceutical preparation according of the invention, and the use of a preparation of the invention for the preparation of a medicament for treatment of type 1 or type 2 diabetes.

### DEFINITIONS

Unless otherwise specified, the term “insulin” as used herein is intended to refer to not only human insulin as such, but also insulin analogues and derivatives. “Human insulin” is well known in the art, and is given below for convenience:

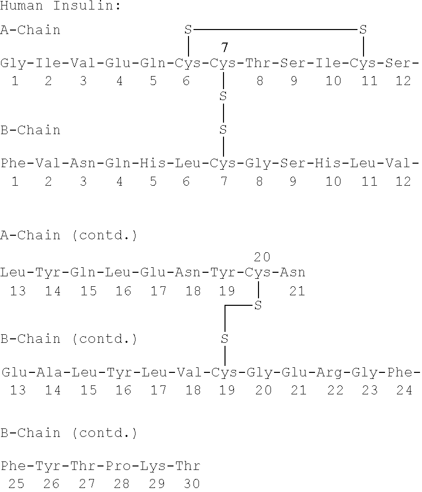

The term “analogue of human insulin” as used herein refers to a polypeptide having the amino acid sequence of human insulin in which one or more amino acids have been deleted and/or replaced by other amino acids, including amino acids not encoded by the genetic code, or comprising additional amino acids, i.e. more than the 51 amino acids of human insulin.

The term “derivative of human insulin” as used herein refers to human insulin or an analogue thereof in which at least one organic substituent is bound to one or more of the amino acids. Non-limiting examples of a “derivative of human insulin” are B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin

The term “chemical stability” as used herein refers to the tendency of an insulin composition to form stable hexamer structures.

In the present context, the unit “U” corresponds to 6 nmol.

The terms “treatment” and “treating” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

The three letter codes and one letter codes for the amino acid residues used herein are those stated in J. Biol. Chem. 243, p. 3558 (1968).

### DESCRIPTION OF THE INVENTION

The present invention relates to rapid-acting analogues of human insulin wherein the amino acid in position B26 is Phe. Such analogues have been found to be particularly useful when used in premixed pharmaceutical preparations where another, long-acting insulin is also present. The monomer insulin molecules of the present invention do not interact with the hexamer formed by the long-acting insulin molecules to the same degree as known fast-acting analogues. As a result, the release profile of insulin after injection of the pre-mixed preparation shows an improved release profile, in that both an rapid initial release as well as a continuing, basal release is observed.

In one embodiment the invention provides analogues of human insulin wherein the amino acid in position B26 is Phe and wherein the amino acid sequence is at least 80% identical to that of human insulin. This and the following analogues wherein the amino acid in position B26 is Phe are termed Group A analogues.

In another embodiment the invention provides an analogue of human insulin wherein the amino acid in position B26 is Phe and which has a half life of less than 3 hours measured in the disappearance assay described herein.

In another embodiment the invention provides an analogue of human insulin having the sequence

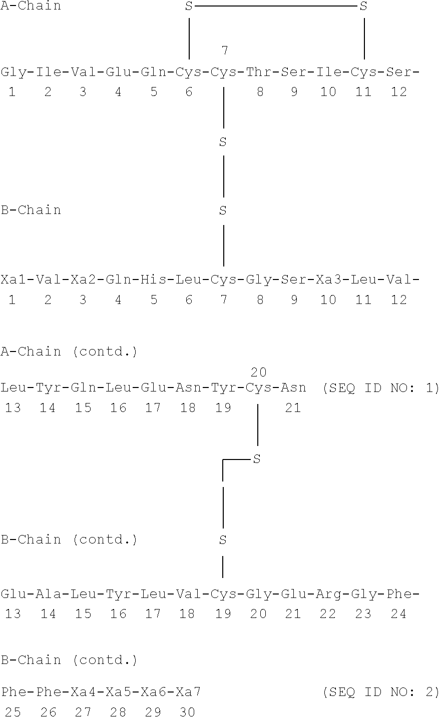

wherein Xa1 is Phe, Glu or —H,

### Xa2 is Asn or Lys,

### Xa3 is His or Asp,

### Xa4 is Thr or Pro,

### Xa5 is Pro, Thr, Lys, Asp or Ile,

### Xa6 is Lys, Thr, Pro or Glu,

### Xa7 is Thr, Lys, Pro, Glu or —OH,

In one embodiment Xa1 is Phe or —H.

In another embodiment Xa1 is Phe.

In another embodiment Xa1 is Glu.

In another embodiment Xa1 is —H.

In another embodiment Xa2 is Asn.

In another embodiment Xa2 is Lys.

In another embodiment Xa3 is His.

In another embodiment Xa3 is Asp.

In another embodiment Xa4 is Thr.

In another embodiment Xa4 is Lys.

In another embodiment Xa5 is Pro, Lys or Asp.

In another embodiment Xa5 is Pro.

In another embodiment Xa5 is Lys.

In another embodiment Xa5 is Asp.

In another embodiment Xa5 is Thr.

In another embodiment Xa5 is Ile.

In another embodiment Xa6 is Lys.

In another embodiment Xa6 is Thr.

In another embodiment Xa6 is Pro.

In another embodiment Xa6 is Glu.

In another embodiment Xa7 is Thr.

In another embodiment Xa7 is Lys.

In another embodiment Xa7 is Pro.

In another embodiment Xa7 is Glu.

In another embodiment Xa7 is —OH.

The situation where Xa1 is —H corresponds to deletion of the amino acid in position B1. Similarly, the situation where Xa7 is —OH corresponds to deletion of the amino acid in position B30.

In another embodiment the invention provides a pharmaceutical preparation comprising
i. A derivative of human insulin
ii. an analogue of human insulin having a fast onset of action, wherein
   Phe(B1) is deleted, or
   Tyr(B26) is replaced with Phe, or
   Phe(B1) is deleted and Tyr(B26) is replaced with Phe.

In one embodiment the invention provides a pharmaceutical preparation wherein the ratio of the derivative of human insulin i. to the analogue of human insulin having a fast onset of action ii. is between 1:99 and 99:1.

In another embodiment the ratio is between 10:90 and 90:10.

In another embodiment the ratio is between 30:70 and 70:30.

In one embodiment the invention provides a pharmaceutical preparation wherein the derivative of human insulin i. is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

In another embodiment the derivative of human insulin i. is B29-N^{ε}-(N-lithocholyl-γ-glutamyl)-des(B30) human insulin.

In another embodiment the derivative of human insulin i. is B29-N^{ε}-myristoyl-des(B30) human insulin.

In one embodiment the invention provides a pharmaceutical preparation wherein the analogue of human insulin ii. is Des(B1) human insulin or Des(B1) Des(B30) human insulin.

In another embodiment the analogue of human insulin ii. is Des(B1) human insulin.

In one embodiment the invention provides a pharmaceutical preparation wherein the analogue of human insulin ii. is an analogue selected from Group A analogues.

In one embodiment the invention provides a pharmaceutical preparation which further comprises a phenolic preservative.

The invention furthermore provides a method of treating type 1 or type 2 diabetes comprising administering to a patient in need thereof a therapeutically effective amount of a pharmaceutical preparation of the invention.

In another embodiment the invention provides the use of a preparation of the invention for the preparation of a medicament for treatment of type 1 or type 2 diabetes.

In another aspect the invention provides an analogue of human insulin wherein the amino acid in position B26 is Phe and wherein the amino acid sequence is at least 80% identical to that of human insulin.

In one embodiment the invention provides an analogue of human insulin wherein the amino acid in position B26 is Phe and which has a time of disappearance of less than 3 hours measured in the disappearance assay described herein.

In one embodiment the invention provides an analogue of human insulin having the sequence

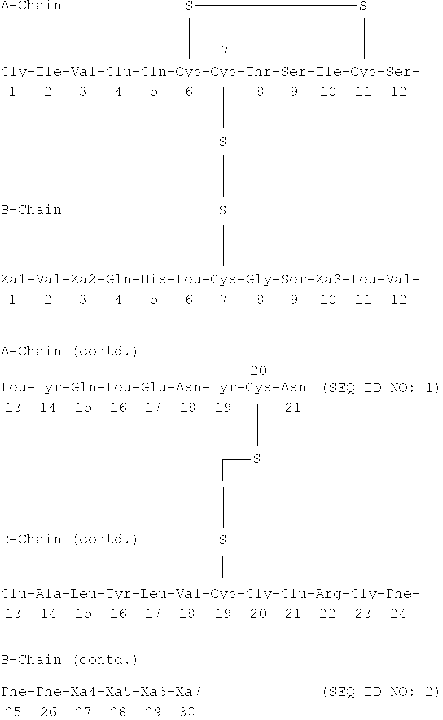

wherein Xa1 is Phe, Glu or —H,

### Xa2 is Asn or Lys,

### Xa3 is Ala, Asp, Glu, Phe, Gly, Ile, Lys, Leu, Met, Asn, Pro, Gln, Arg, Ser, Thr, Val, Trp, or Tyr,

### Xa4 is Thr or Pro,

### Xa5 is Pro, Thr, Lys, Asp or Ile,

### Xa6 is Lys, Thr, Pro or Glu,

### Xa7 is Thr, Lys, Pro, Glu or —OH,

In one embodiment Xa1 is Phe or —H.

In one embodiment Xa1 is Phe.

In one embodiment Xa1 is Glu.

In one embodiment Xa1 is —H.

In one embodiment Xa2 is Asn.

In one embodiment Xa2 is Lys.

In one embodiment Xa3 is Ala, Asp, Glu, Phe, Ile, Lys, Leu, Met, Asn, Gln, Arg, Ser, Thr, Val, Trp, or Tyr.

In one embodiment Xa3 is Ala, Thr, Ser, Asn or Gln.

In one embodiment Xa3 is Ala, Thr, or Ser.

In one embodiment Xa3 is Ala.

In one embodiment Xa4 is Thr.

In one embodiment Xa4 is Lys.

In one embodiment Xa5 is Pro, Lys or Asp.

In one embodiment Xa5 is Pro.

In one embodiment Xa5 is Lys.

In one embodiment Xa5 is Asp.

In one embodiment Xa5 is Thr.

In one embodiment Xa5 is Ile.

In one embodiment Xa6 is Lys.

In one embodiment Xa6 is Thr.

In one embodiment Xa6 is Pro.

In one embodiment Xa6 is Glu.

In one embodiment Xa7 is Thr.

In one embodiment Xa7 is Lys.

In one embodiment Xa7 is Pro.

In one embodiment Xa7 is Glu.

In one embodiment Xa7 is —OH.

In one embodiment the invention provides a pharmaceutical preparation comprising
i. A derivative of human insulin
ii. an analogue of human insulin having a fast onset of action, wherein
   Phe(B1) is deleted, or
   Tyr(B26) is replaced with Phe, or
   Phe(B1) is deleted and Tyr(B26) is replaced with Phe.

In one embodiment the ratio of the derivative of human insulin i. to the analogue of human insulin having a fast onset of action ii. is between 1:99 and 99:1.

In one embodiment the ratio is between 10:90 and 90:10.

In one embodiment the ratio is between 30:70 and 70:30.

In one embodiment the derivative of human insulin i. is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

In one embodiment the derivative of human insulin i. is B29-N^{ε}-(N-lithocholyl-γ-glutamyl)-des(B30) human insulin.

In one embodiment the derivative of human insulin i. is B29-N^{ε}-myristoyl-des(B30) human insulin.

In one embodiment the analogue of human insulin ii. is Des(B1) human insulin or Des(B1) Des(B30) human insulin.

In one embodiment the analogue of human insulin ii. is Des(B1) human insulin.

In one embodiment the analogue of human insulin ii. is an analogue according to any one of the embodiments above.

In one embodiment the invention provides a pharmaceutical preparation according to any one of the embodiments above which further comprises a phenolic preservative.

In one embodiment the invention provides a method of treating type 1 or type 2 diabetes comprising administering to a patient in need thereof a therapeutically effective amount of a pharmaceutical preparation according to any one of the embodiments above.

In one embodiment the invention provides the use of a preparation according to any one of the embodiments above for the preparation of a medicament for treatment of type 1 or type 2 diabetes.

### Pharmaceutical Compositions

Insulin preparations of the invention are usually administered from multi-dose containers where a preservative effect is desired. In one embodiment the preservative may be a phenolic molecules. The phenolic molecules in the insulin preparation may be selected from the group consisting of phenol, m-cresol, chloro-cresol, thymol, m-chlor-phenol, resorcinole, 7-hydroxyindole or any mixture thereof.

In one embodiment of the invention 0.5 to 5.0 mg/ml of phenolic compound may be employed.

In another embodiment of the invention 0.6 to 5.0 mg/ml of m-cresol may be employed.

In another embodiment of the invention 0.5 to 5.0 mg/ml of phenol may be employed.

In another embodiment of the invention 1.4 to 5.0 mg/ml of phenol may be employed.

In another embodiment of the invention 0.5 to 5.0 mg/ml of a mixture of m-cresol or phenol may be employed.

In another embodiment of the invention 1.4 to 5.0 mg/ml of a mixture of m-cresol or phenol may be employed.

The pharmaceutical preparation may further comprise a buffer substance, such as a TRIS, phosphate, glycine or glycylglycine (or another zwitterionic substance) buffer, an isotonicity agent, such as NaCl, glycerol, mannitol and/or lactose

The pharmaceutical preparation may further comprise physiologically acceptable agents that increase the viscosity of the pharmaceutical preparation. Thus, the pharmaceutical preparation according to the invention may furthermore comprise an agent which increases the viscosity, such as polyethylene glycol, polypropylene glycol, copolymers thereof, dextrans and/or polylactides.

In a particular embodiment the insulin preparation of the invention comprises between 0.001% by weight and 1% by weight of a non-ionic surfactant, for example tween 20 or Poloxamer 188.

The insulin preparation of the present invention may have a pH value in the range of 3.5 to 8.5, more preferably 7.1 to 7.9.

### Combination Treatment

The invention furthermore relates to treatment of a patient in which the Group A insulin analogue of the invention and/or a pharmaceutical preparation of the invention, i.e. a preparation comprising both a Group A insulin analogue and a derivative of human insulin such as B29-N^{ε}-(N-lithocholyl-γ-glutamyl)-des(B30) human insulin, is combined with another form of treatment.

In one aspect of the invention, treatment of a patient with the pharmaceutical preparation of the invention is combined with diet and/or exercise.

In another aspect of the invention the pharmaceutical preparation of the invention is administered in combination with one or more further active substances in any suitable ratios. Such further active substances may e.g. be selected from antiobesity agents, antidiabetics, anti-hypertensive agents, agents for the treatment of complications resulting from or associated with diabetes and agents for the treatment of complications and disorders resulting from or associated with obesity.

Thus, in a further aspect of the invention the pharmaceutical preparation of the invention may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, MC3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, 63 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, mixed serotonin and noradrenergic compounds, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TR β agonists, AGRP (Agouti related protein) inhibitors, H3 histamine antagonists, opioid antagonists (such as naltrexone), exendin-4, GLP-1 and ciliary neurotrophic factor.

In one embodiment of the invention the antiobesity agent is leptin.

In another embodiment the antiobesity agent is dexamphetamine or amphetamine.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

In still another embodiment the antiobesity agent is phendimetrazine, diethylpropion, fluoxetine, bupropion, topiramate or ecopipam.

The orally active hypoglycemic agents comprise imidazolines, sulphonylureas, biguanides, meglitinides, oxadiazolidinediones, thiazolidinediones, insulin sensitizers, insulin secretagogues such as glimepride, α-glucosidase inhibitors, agents acting on the ATP-dependent potassium channel of the β-cells eg potassium channel openers such as those disclosed in WO 97/26265, WO 99/03861 and WO 00/37474 (Novo Nordisk A/S) which are incorporated herein by reference, or mitiglinide, or a potassium channel blocker, such as BTS-67582, nateglinide, glucagon antagonists such as those disclosed in WO 99/01423 and WO 00/39088 (Novo Nordisk A/S and Agouron Pharmaceuticals, Inc.), which are incorporated herein by reference, GLP-1 agonists such as those disclosed in WO 00/42026 (Novo Nordisk A/S and Agouron Pharmaceuticals, Inc.), which are incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antilipidemic agents, compounds lowering food intake, PPAR (peroxisome proliferator-activated receptor) and RXR (retinoid X receptor) agonists, such as ALRT-268, LG-1268 or LG-1069.

In a further embodiment of the invention the pharmaceutical preparation of the invention is administered in combination with a sulphonylurea e.g. tolbutamide, chlorpropamide, tolazamide, glibenclamide, glipizide, glimepiride, glicazide or glyburide.

In another embodiment of the invention the pharmaceutical preparation of the invention is administered in combination with a biguanide, e.g. metformin.

In yet another embodiment of the invention the pharmaceutical preparation of the invention is administered in combination with a meglitinide eg repaglinide or nateglinide.

In still another embodiment of the invention the pharmaceutical preparation of the invention is administered in combination with a thiazolidinedione insulin sensitizer, e.g. troglitazone, ciglitazone, pioglitazone, rosiglitazone, isaglitazone, darglitazone, englitazone, CS-011/CI-1037 or T 174 or the compounds disclosed in WO 97/41097, WO 97/41119, WO 97/41120, WO 00/41121 and WO 98/45292 (Dr. Reddy's Research Foundation), which are incorporated herein by reference.

In still another embodiment of the invention the pharmaceutical preparation of the invention may be administered in combination with an insulin sensitizer, e.g. such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313, WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In a further embodiment of the invention the pharmaceutical preparation of the invention is administered in combination with an α-glucosidase inhibitor, e.g. voglibose, emiglitate, miglitol or acarbose.

In another embodiment of the invention the pharmaceutical preparation of the invention is administered in combination with an agent acting on the ATP-dependent potassium channel of the β-cells, e.g. tolbutamide, glibenclamide, glipizide, glicazide, BTS-67582 or repaglinide.

In yet another embodiment of the invention the pharmaceutical preparation of the invention may be administered in combination with nateglinide.

In still another embodiment of the invention the pharmaceutical preparation of the invention is administered in combination with an antilipidemic agent, e.g. cholestyramine, colestipol, clofibrate, gemfibrozil, lovastatin, pravastatin, simvastatin, probucol or dextrothyroxine.

In another aspect of the invention, the pharmaceutical preparation of the invention is administered in combination with more than one of the above-mentioned compounds, e.g. in combination with metformin and a sulphonylurea such as glyburide; a sulphonylurea and acarbose; nateglinide and metformin; acarbose and metformin; a sulphonylurea, metformin and troglitazone; metformin and a sulphonylurea; etc.

Furthermore, the pharmaceutical preparation of the invention may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. The pharmaceutical preparation of the invention may also be combined with NEP inhibitors such as candoxatril.

Further reference can be made to Remington: The Science and Practice of Pharmacy, 19^{th} Edition, Gennaro, Ed., Mack Publishing Co., Easton, Pa., 1995.

It should be understood that any suitable combination of the compounds according to the invention with diet and/or exercise, one or more of the above-mentioned compounds and optionally one or more other active substances are considered to be within the scope of the present invention.

Whenever a Group A analogue of this invention is combined with another form of treatment, this administration can be simulataneous or sequential, in a manner effective to result in their combined actions within the subject treated. In one embodiment, a Group A analogue may be administered in combination with long-acting derivatives of human insulin as described above, either as a pre-mixed preparation, or by substantially simultaneous administraton of two separate preparations, or by sequential administration, i.e. administrations may be separated in time. The agents would be provided in amounts effective and for periods of time effective to result in their combined presence and their combined actions. The administration of a Group A analogue of the invention may precede, or follow, the other form of treatment by, e.g., intervals ranging from minutes to weeks and months.

The compositions of the present invention may be administered orally, parenterally, by inhalation spray, topically, rectally, nasally, buccally, ophthalmiccaly, vaginally or via an implanted reservoir. In one embodiment, the pharmaceutical compositions according to the invention may be used for parenteral administration, such as subcutaneous, intramuscular, intrathecal, intravenous, intradermal, intraspinal or intrasternal administration.

It will be appreciated that the preferred route of administration will depend on the general condition and age of the subject to be treated, the nature of the condition to be treated and the active ingredient chosen.

Suitable administration forms include sterile aqueous and non-aqueous injectable solutions, dispersions, suspensions or emulsions as well as sterile powders to be reconstituted in sterile injectable solutions or dispersions prior to use. Depot injectable preparations are also contemplated as being within the scope of the present invention.

Other suitable administration forms include ophthalmic preparations such as eye drops and eye ointments and topical preparations such as wound dressings.

In one embodiment the preparations of the invention are used in connection with insulin pumps. The insulin pumps may be prefilled and disposable, or the insulin preparations may be supplied from a reservoir which is removable. Insulin pumps may be skin-mounted or carried, and the path of the insulin preparation from the storage compartment of the pump to the patient may be more or less tortuous. Non-limiting examples of insulin pumps are disclosed in U.S. Pat. No. 5,957,895, U.S. Pat. No. 5,858,001, U.S. Pat. No. 4,468,221, U.S. Pat. No. 4,468,221, U.S. Pat. No. 5,957,895, U.S. Pat. No. 5,858,001, U.S. Pat. No. 6,074,369, U.S. Pat. No. 5,858,001, U.S. Pat. No. 5,527,288, and U.S. Pat. No. 6,074,369.

In another embodiment the preparations of the invention are used in connection with pen-like injection devices, which may be prefilled and disposable, or the insulin preparations may be supplied from a reservoir which is removable. Non-limiting examples of pen-like injection devices are FlexPen®, InnoLet®, InDuo™, Innovo®.

In a further embodiment preparations of the invention are used in connection with devices for pulmonary administration of aqueous insulin preparations, a non-limiting example of which is the AerX® device.

### Examples

### Disappearance Assay

Rapid-acting analogues of human insulin of the invention were prepared by site-directed mutagenesis as described in Brange et al., Nature vol 333 page 679-682 (1988).

Derivatives of human insulin having a protracted action were made by acylation of biosynthetic des(B30) human insulin in position Lys^{B29}N^{e} by tetradecanoic acid and a series of cholic acid derivatives using conventional peptide chemistry as described in WO 99/21888.

Insulin and insulin analogues for pharmacokinetic experiments were prepared as preparations containing 600 nmol of insulin per ml and 2-2.5 Zn²⁺ per hexamer, 1,5% glycerol and 0.3% phenol. Insulin preparations for pharmacokinetic experiments were labelled by ⁶⁵Zn²⁺ or by iodinationation of Tyr^{A14} of the appropriate analogues by ¹²⁵I₂ (see Jorgensen, K H, Larsen, U D: Homogeneous mono-125I-insulins. Preparation and characterization of Mono-125I-(Tyr A14)- and Mono-125I-(Tyr A19)-insulin. DIABETOLOGIA 19:546-554, 1980) and NHP insulin was labelled by Tyr^{A14}(¹²⁵I-human insulin).

Formulated preparations of insulin analogues labelled in position Tyr^{A14} by ¹²⁵I or by ⁶⁵Zn²⁺ were injected subcutaneously in pigs as previously described (Ribel, U., Jøorgensen, K, Brange, J, and Henriksen, U.: The pig as a model for subcutaneous insulin absorption in man. Serrano-Rios, M and Lefèbvre, P. J. 891-896. 1985. Amsterdam; New York; Oxford, Elsevier Science Publishers. 1985). The disappearance of the radioactive label from the site of subcutaneous injection was monitored using a modification of the traditional external gamma-counting method (Ribel, U.: Subcutaneous absorption of insulin analogues. Berger, M. and Gries, F. A. 70-77. 1993. Stuttgart; New York, Georg Thime Verlag). With this modified method it was possible to measure continuously the disappearance of radioactivity from a subcutaneous depot for several days using cordless portable device (Scancys Laboratorieteknik, Vaerløse, DK-3500). The measurements were performed at 1-min intervals, and the counting values were corrected for background activity. An insulin dose of 60 nmol (equal to 10 units of human insulin) was used and each pig received both a test analogue and tetradecanoyl des(B30) human insulin in separate depots. Results are reported as the half-life of the insulin species in the subcutaneous depot, i.e. as the time measured to reduce the radioactivity to one half of the initial level.

The following insulin analogues were tested in a preparation also containing protracted insulin B29-N^{ε}-(N-lithocholyl-γ-glutamyl)-des(B30) human insulin (in the following table termed ProtIns) in the amounts indicated in the table below:

Analogue 1: B10Asp B26Phe DesB30 human insulin.
Analogue 2: B10Asp B26Phe B28Asp DesB30 human insulin.

For comparison B28Asp human insulin was tested.

| | | | | | | Injec- | |
|---|---|---|---|---|---|---|---|
| | concentra- | Zn/ | | Preservative | | tion | T½ |
| Insulin | tion | hexamer | Isotonicity agent | Phenol/Cresol | phosphate | volume | (hours) |
| Analogue 1 + | 180 + 420 μM | 2 | Glycerol 1.6% | 16 mM | 7 mM | 100 μl | 1.14 |
| ProtIns | | | NaCl 20 mM | | pH 7.5 | | |
| Analogue 2 + | 180 + 420 μM | 2 | Glycerol 1.6% | 16 mM | 7 mM | 100 μl | 0.84 |
| ProtIns | | | NaCl 20 mM | | pH 7.5 | | |
| B28Asp human | 180 μM | 2 | Glycerol 1.6% | 16 mM | 7 mM | 100 μl | 0.86 |
| insulin | | | NaCl 20 mM | | pH 7.5 | | |

1. An analogue of human insulin having an amino acid sequence wherein the amino acid in position B26 is Phe and wherein the amino acid sequence is at least 80% identical to that of human insulin.

2. An analogue of human insulin having an amino acid sequence wherein the amino acid in position B26 is Phe and which has a time of disappearance of less than 3 hours measured in a disappearance assay.

3. An analogue of human insulin having a sequence 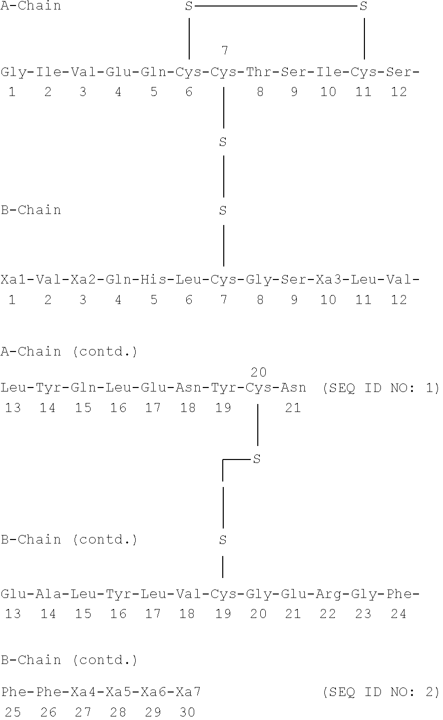
wherein Xa1 is Phe, Glu or —H,
Xa2 is Asn or Lys,
Xa3 is Ala, Asp, Glu, Phe, Gly, Ile, Lys, Leu, Met, Asn, Pro, Gln, Arg, Ser, Thr, Val, Trp, or Tyr,
Xa4 is Thr or Pro,
Xa5 is Pro, Thr, Lys, Asp or Ile,
Xa6 is Lys, Thr, Pro or Glu,
Xa7 is Thr, Lys, Pro, Glu or —OH,

4. An analogue of human insulin according to claim 3 wherein Xa1 is Phe or —H.

5. An analogue of human insulin according to claim 4 wherein Xa1 is Phe.

6. An analogue of human insulin according to claim 3 wherein Xa1 is Glu.

7. An analogue of human insulin according to claim 4 wherein Xa1 is —H.

8. An analogue of human insulin according to claim 3 wherein Xa2 is Asn.

9. An analogue of human insulin according to claim 3 wherein Xa2 is Lys.

10. An analogue of human insulin according to claim 3 wherein Xa3 is Ala, Asp, Glu, Phe, Ile, Lys, Leu, Met, Asn, Gln, Arg, Ser, Thr, Val, Trp, or Tyr.

11. An analogue of human insulin according to claim 10 wherein Xa3 is Ala, Thr, Ser, Asn or Gln.

12. An analogue of human insulin according to claim 11 wherein Xa3 is Ala, Thr, or Ser.

13. An analogue of human insulin according to claim 3 wherein Xa3 is Ala.

14. An analogue of human insulin according to claim 3 wherein Xa4 is Thr.

15. An analogue of human insulin according to claim 3 wherein Xa4 is Lys.

16. An analogue of human insulin according to claim 3 wherein Xa5 is Pro, Lys or Asp.

17. An analogue of human insulin according to claim 16 wherein Xa5 is Pro.

18. An analogue of human insulin according to claim 16 wherein Xa5 is Lys.

19. An analogue of human insulin according to claim 16 wherein Xa5 is Asp.

20. An analogue of human insulin according to claim 3 wherein Xa5 is Thr.

21. An analogue of human insulin according to claim 3 wherein Xa5 is Ile.

22. An analogue of human insulin according to claim 3 wherein Xa6 is Lys.

23. An analogue of human insulin according to claim 3 wherein Xa6 is Thr.

24. An analogue of human insulin according to claim 3 wherein Xa6 is Pro.

25. An analogue of human insulin according to claim 3 wherein Xa6 is Glu.

26. An analogue of human insulin according to claim 3 wherein Xa7 is Thr.

27. An analogue of human insulin according to claim 3 wherein Xa7 is Lys.

28. An analogue of human insulin according to claim 3 wherein Xa7 is Pro.

29. An analogue of human insulin according to claim 3 wherein Xa7 is Glu.

30. An analogue of human insulin according to claim 3 wherein Xa7 is —OH.

31. A pharmaceutical preparation comprising
i. A derivative of human insulin
ii. an analogue of human insulin having a fast onset of action, wherein
Phe(B1) is deleted, or
Tyr(B26) is replaced with Phe, or
Phe(B1) is deleted and Tyr(B26) is replaced with Phe.

32. A pharmaceutical preparation according to claim 31 wherein the ratio of the derivative of human insulin to the analogue of human insulin having a fast onset of action is between 1:99 and 99:1.

33. A pharmaceutical preparation according to claim 32 wherein the ratio is between 10:90 and 90:10.

34. A pharmaceutical preparation according to claim 33 wherein the ratio is between 30:70 and 70:30.

35. A pharmaceutical preparation according to claim 31 wherein the derivative of human insulin is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

36. A pharmaceutical preparation according to claim 35 wherein the derivative of human insulin is B29-N^{ε}-(N-lithocholyl-γ-glutamyl)-des(B30) human insulin.

37. A pharmaceutical preparation according to claim 35 wherein the derivative of human insulin is B29-N^{ε}-myristoyl-des(B30) human insulin.

38. A pharmaceutical preparation according to claim 31 wherein the analogue of human insulin is Des(B1) human insulin or Des(B1) Des(B30) human insulin.

39. A pharmaceutical preparation according to claim 38 wherein the analogue of human insulin is Des(B1) human insulin.

40. A pharmaceutical preparation according to claim 31 wherein the analogue of human insulin is an analogue according to claim 1.

41. A pharmaceutical preparation according to claim 31 wherein the analogue of human insulin is an analogue according to claim 2.

42. A pharmaceutical preparation according to claim 31 wherein the analogue of human insulin is an analogue according to claim 3.

43. A pharmaceutical preparation according to claim 31 which further comprises a phenolic preservative.

44. A method of treating type 1 or type 2 diabetes, the method comprising administering to a patient in need thereof a therapeutically effective amount of a pharmaceutical preparation according to claim 31.


SEQUENCE LISTING (2 sequences)

SEQ ID NO: 1 - protein, 21 aa, Homo sapiens
GIVEQCCTSICSLYQLENYCN

SEQ ID NO: 2 - protein, 30 aa, Homo sapiens
  MISC_FEATURE (1)..(1): Xaa can be Phe, Glu
XVXQHLCGSXLVEALYLVCGERGFFFXXXX